(19) United States
(12) Patent Application Publication
(10) Pub. No.: US 2022/0162645 A1
(43) Pub. Date: May 26, 2022

(54) CELL PORATION AND TRANSFECTION APPARATUSES

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US); Diane R. Hammerstad, Corvallis, OR (US)

(73) Assignee: Hewlett-Packard Development Company, L.P., Spring, TX (US)

(21) Appl. No.: 17/414,641

(22) PCT Filed: Jul. 25, 2019
(86) PCT No.: PCT/US2019/043391

§ 371 Date: Jun. 16, 2021

(51) Int. Cl. C12N 15/87; C12M 1/42; C12M 1/34; C12M 1/36; B01L 3/00; G01V 3/02; G01N 15/14; G01N 27/02

(52) U.S. Cl. CPC C12N 15/87; C12M 35/00; C12M 41/12; C12M 41/48; B01L 3/502761; G01N 2015/0065; G01V 3/02; G01N 15/1434; G01N 27/02; B01L 2200/0647; B01L 3/502746

(57) ABSTRACT

In example implementations, an apparatus is provided. The apparatus includes a channel, a thermal inkjet (TIJ) resistor, and a transfection chamber. The TIJ resistor is to apply heat to a cell in the channel to porate the cell. The transfection chamber is to store a reagent to be inserted into the cell after the cell is porated.

### BACKGROUND

Cell transfection may be used for research and production of certain biological products, such as synthetic proteins, genetically modified organisms, and the like. Cell transfection includes creating pores in a cell membrane of a cell and inserting a foreign material into the cell.

### BRIEF DESCRIPTION OF THE DRAWINGS

FIG. 1 is a block diagram of an example system to provide cell poration and transfection of the present disclosure;

FIG. 2 is a block diagram of a cross-sectional view of an apparatus with thermal inkjet (TIJ) resistors to provide thermal cell poration and transfection of the present disclosure;

FIG. 3 is a block diagram of a top view of different arrangements of porating chambers of the apparatus of the present disclosure;

FIG. 4 is a block diagram of examples of cross-sectional views of different arrangements of the TIJ resistor of the present disclosure;

FIG. 5 is a block diagram of an example apparatus to provide thermal cell poration and transfection having a cell monitor and a feedback loop of the present disclosure;

FIG. 6 is a block diagram of an example apparatus to provide thermal cell poration and transfection having multiple parallel transfection chambers of the present disclosure;

FIG. 7 is a block diagram of a top view of an apparatus to provide thermal cell poration and transfection with a separator; and

FIG. 8 is a flow chart of an example method for poration cells for transfection of the present disclosure.

### DETAILED DESCRIPTION

Examples described herein provide a system and apparatus for poration of cells for transfection. As noted above, transfection of cells can be used for research or production of certain biological products. Transfection allows the behavior of the cell to be changed. For example, by diffusing a reagent, such as a particular DNA along with proteins that incorporate the DNA, into a cell's genome, the cell's genome may be altered to create a genetically modified organism.

Some systems to perform the poration and transfection can potentially introduce contamination. Some methods for poration of cells use expensive tooling or may not provide sufficient control of the amount of material that is transfected into a cell. Other methods may also not allow for transfection with microscopic amounts of fluids in single cells.

Examples herein provide a system and apparatus for poration of cells for transfection. The apparatus may apply thermal energy to porate the cell. The thermal energy may be provided by a TIJ resistor. The design of the present apparatus can perform poration and transfection of cells without contamination. In addition, the TIJ resistor allows for single cell precision, which may provide better transfection yield and better survivability.

In one example, the system may also provide cell monitoring and a feedback control loop. For example, sensors may be implemented to detect the presence of a cell to control operation of the energy source. As a result, the energy source may be activated and deactivated based on whether cells are present in the apparatus.

In addition, the feedback control loop may detect if the cells have been porated for transfection. If not, the cells may be returned to the porating chamber to repeat the poration process. Thus, the system may ensure that the cells are properly porated.

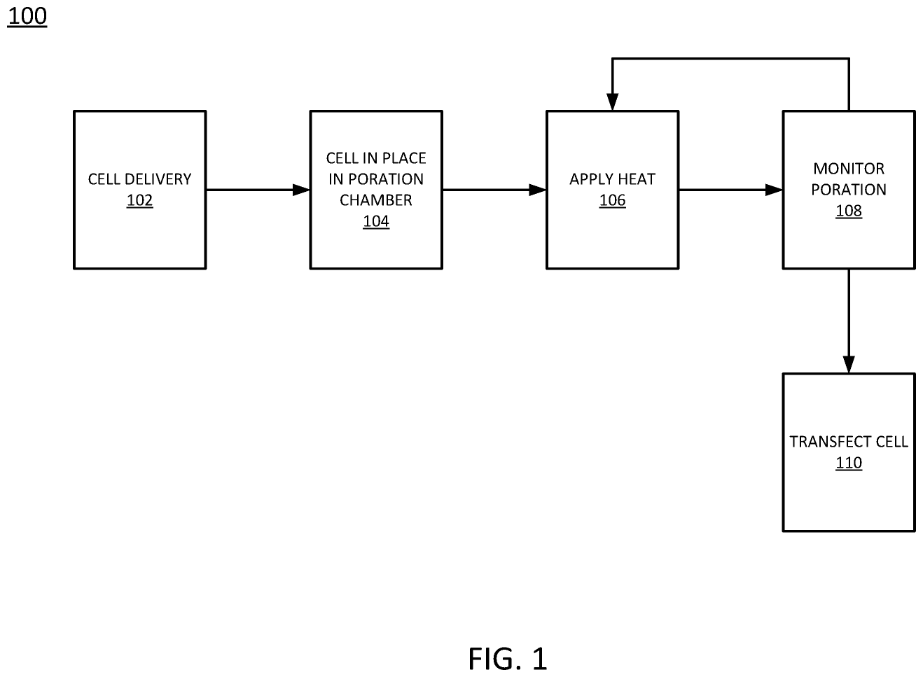

FIG. 1 illustrates an example block diagram of a system 100 of the present disclosure. The block diagram of the system 100 provides an overview of how the apparatus described herein may operate. Different examples of the structure to perform the functions of each block of the system 100 are illustrated and discussed in further details below.

At block 102 cell delivery may be provided. For example, the cells may be stored in a reservoir in a fluid. The cells may be delivered via a pump. At block 104, the cells may be placed in a poration chamber and positioned to receive heat.

At block 106, heat may be applied to the cell to porate the cell. The heat may create pores in the membrane of the cell. The pores may allow the cell to be transfected with a reagent. At block 108, the poration of the cell may be monitored. For example, a monitoring system may monitor whether or not pores are being properly formed in the cell. Based on the monitoring, the amount of heat that is applied to the cell can be adjusted as part of a feedback loop.

If the cell is successfully porated, the cell may be released into a transfection chamber. At block 110, the cell may be transfected with a reagent. The reagent may be any type of molecule that can be injected into the cell. The type of reagent may be a function of a purpose of the transfection. For example, the reagent may be used to reprogram the cell, mutate the cell, or create new cells. The reagent may include DNA, RNA, proteins, nanoparticles, and the like.

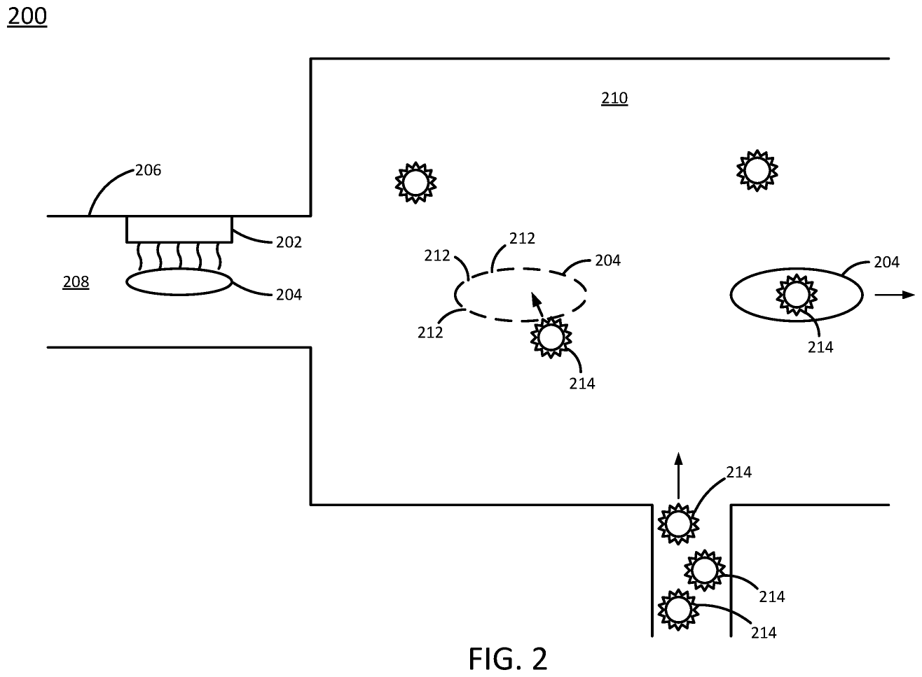

FIG. 2 illustrates a block diagram of a cross-sectional view of an apparatus 200 with thermal inkjet (TIJ) resistors 202 to provide thermal cell poration and transfection of the present disclosure. In one example, the apparatus 200 may include a poration chamber 206. A cell 204 may be provided through a channel 208. The TIJ resistor 202 may generate heat that is applied to the cell 204 to porate the cell 204.

The TIJ resistor 202 may comprise a circuit that includes a resistor. Current can be controlled through the resistor via a series of switches. When the switches are activated to complete the circuit, current is allowed to flow through the resistor and the resistor may generate heat. When the switches are deactivated, the circuit may be open to prevent current from flowing through the resistor. When no current flows through the resistor no heat is generated.

In one example, the TIJ resistors 202 may be operated in a pulsing fashion. For example, the TIJ resistors 202 may be periodically activated and deactivated. The pulse frequency may be high enough that when the cell 204 goes through the heating zone, the cell statistically encounters several such pulses. In one example, the flow rate or velocity of the cell 204 may be less than or equal to the length of the TIJ resistor 202 times the frequency of the heater pulse. The flow rate of the cell 204 may be the average velocity of the cells 204 in the channel 208.

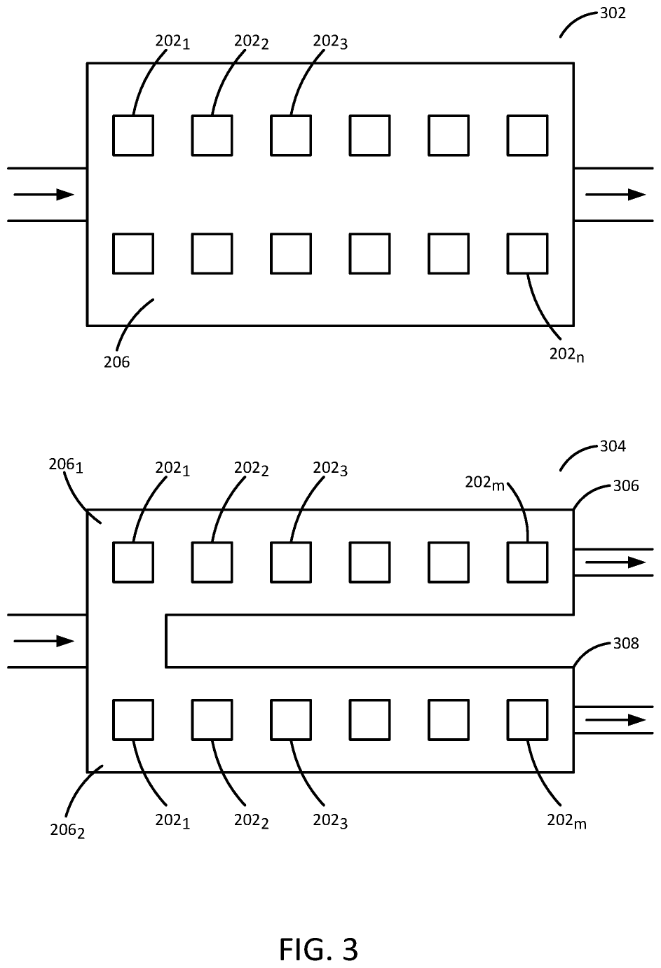

In another example, the TIJ resistors 202 may be operated in pulses that may be synchronized with the travel of the cells 204. The pulses may be synchronized so that each cell 204 receives a user specified number of pulses. In both modes of operation, the TIJ resistors 202 may be arranged in series (as illustrated in FIG. 3 and discussed below) to increase the overall amount of transfection so that the cell 204 has time to recover as the cell 204 travels between the TIJ resistors 202.

The TIJ resistor 202 may be formed on a silicon substrate that is in the channel 208 of the poration chamber 206. In one example, the channel 208 may include a plurality of TIJ resistors 202 in a variety of different arrangements. FIG. 3 illustrates a top view of different arrangements 302 and 304 of the TIJ resistors 202.

In one example, the arrangement 302 may include arrays of TIJ resistors 202₁ to 202ₙ in a poration chamber 206. For example, the TIJ resistors 202 may be arranged in parallel lines or arrays through the poration chamber 206. Thus, the cells 204 may be heated by one of the TIJ resistors 202 as the cells 204 flow through the poration chamber 206. Although two arrays of TIJ resistors 202 are illustrated in the arrangement 302, it should be noted that any number of arrays may be implemented.

In one example, the arrangement 304 may include an array of TIJ resistors 202₁ to 202ₘ in parallel poration chambers 206₁ and 206₂. For example, the array of TIJ resistors may be physically separated into different parallel poration chambers 206. The cells 204 may be heated by one of the TIJ resistors 202 as the cells 204 flow through the respective poration chambers 206. Although two parallel poration chambers 206₁ and 206₂ are illustrated in the arrangement 304, it should be noted that any number of parallel poration chambers 206 may be implemented.

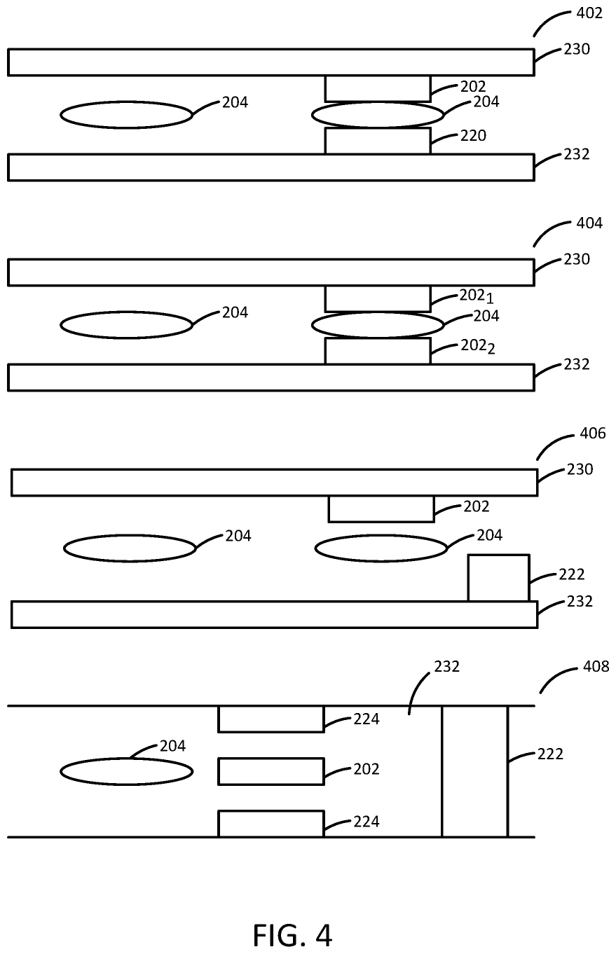

FIG. 4 illustrates different example arrangements 402, 404, 406, and 408 of the TIJ resistor 202 inside of the poration chamber 206. In the arrangement 402, the channel 208 may include a top wall 230 and a bottom wall 232. The TIJ resistor 202 may be coupled to, or formed on, the top wall 230. A raised surface 220 may be coupled to the bottom wall 232. The raised surface 220 may be formed across the width of the channel 208.

In the arrangement 402, the raised surface 220 may be aligned with and located below the TIJ resistor 202 to constrict the cell 204. As shown in the arrangement 402, as the cell 204 flows through the channel 208, the cell 204 may be “squeezed” between the TIJ resistor 202 and the raised surface 220. As a result, the velocity of the cell 204 may be reduced to allow the cell 204 to spend more time below the TIJ resistor 202 to receive heat generated by the TIJ resistor 202.

In addition, the space between the TIJ resistor 202 and the raised surface 220 may be enough for a single cell 204 to flow through between the TIJ resistor 202 and the raised surface 220. Thus, the TIJ resistor 202 may heat one cell 204 at a time.

In the arrangement 404, a first TIJ resistor 202₁ may be located on the top wall 230 and a second TIJ resistor 202₂ may be located on the bottom wall 232. The first TIJ resistor 202₁ and the second TIJ resistor 202₂ may be aligned with one another.

As the cell 204 flows through the channel 208, the cell 204 may be “squeezed” between the TIJ resistors 202₁ and 202₂ to reduce the velocity of the cell 204. In addition, the cell 204 may be more evenly heated by the combination of the TIJ resistors 202₁ and 202₂.

In the arrangement 406, the TIJ resistor 202 may be located on the top wall 230. The bottom wall 232 may include a raised surface 222. The raised surface may be located downstream from the TIJ resistor 202. Thus, the raised surface 222 may temporarily block or slow down the movement of the cell 204 such that the cell 204 may be heated by the TIJ resistor 202.

The arrangement 408 illustrates a top view of the channel 208. In the arrangement 408, the TIJ resistor 202 may be located on the top wall 230 (not shown in the arrangement 408). The raised surface 222 may be located downstream from the TIJ resistor 202 on the bottom surface 232.

In one example, the arrangement 408 may also include side walls 224. The side walls 224 may also be formed on the bottom surface 232 or the side walls of the channel 208. The TIJ resistor 202 may be located between the side walls 224. The raided surface 222 and the sidewalls 224 may reduce the velocity of the cell 204 as it moves through the channel 208. For example, the sidewalls 224 may constrict the cell 204 as the cell 204 moves between the sidewalls 224.

In addition, the sidewalls 224 may align the cell 204 with the TIJ resistor 202. Thus, the TIJ resistor 202 heat the cell 204 more efficiently to porate the cell 204.

Referring back to FIG. 2, the apparatus 200 may include a transfection chamber 210. In one example, the transfection chamber 210 may be larger in size than the channel 208. For example, the transfection chamber 210 may be larger in all directions (e.g., X, Y, and Z directions). The transfection chamber 210 may include at least one inlet channel to feed the reagent 214 into the transfection chamber 210. The inlet channel may be vias from layers below or above the transfection chamber, external ejectors, and the like.

The transfection chamber 210 may store a reagent 214. As noted above, the reagent 214 may be DNA, RNA, proteins, nanoparticles, and the like. The reagent 214 may be inserted into the cell 204 after the cell 204 is porated. For example, the cell 204 may be kept in the transfection chamber 210 for an appropriate amount of time to allow the reagent 214 to enter the cell 204. The flow of the cell 204 into the transfection chamber 210 and the flow of the reagent 214 into the transfection chamber 210 may allow the reagent to interact with the cell 204 and enter the cell 204.

For example, pores 212 may be formed in the cell 204 after the cell 204 is porated. The cell 204 may flow through to the transfection chamber 210 where a reagent 214 may be inserted into the cell 204. After the reagent 214 is inserted into the cell 204, the pores 212 in the membrane of the cell 204 may be allowed to heal, as shown in FIG. 2.

In one example, the transfection chamber 210 may be close enough to the TIJ resistor heater 202 or the poration chamber 206 such that self-healing does not occur before the reagent 214 is inserted into the cell 204. In one example, based on a self-healing time between 10-100 milliseconds and a flow rate of 1-10 centimeters per second, the distance between the poration chamber 206 and the transfection chamber 210 may be between 100-1000 microns apart.

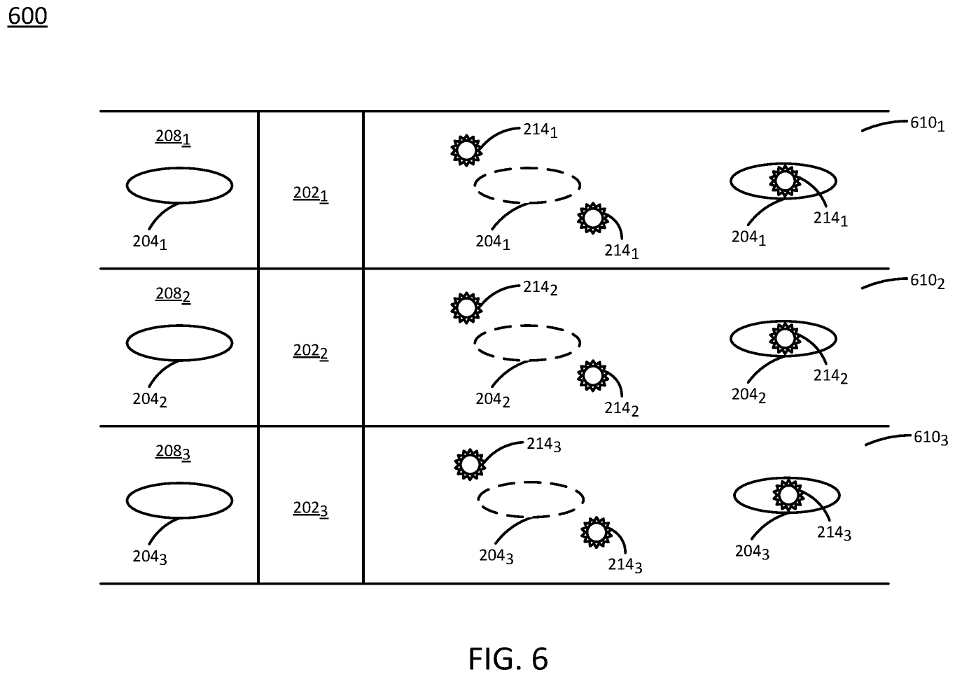

The transfection chamber 210 may include a variety of different arrangements. FIG. 6 illustrates an example arrangement 600 that includes parallel transfection chambers 610₁- 610₃. For example, the cells 204 may be sorted into different channels 208₁-208₃. Each channel 208₁-208₃ may include a respective TIJ resistor 202₁ to 202₃. The cells 204₁-204₃ may flow through the respective channels 208₁-208₃ and be heated by the respective TIJ resistors 202₁ to 202₃.

Each transfection chamber 610₁- 610₃ may include a different type of reagent 214₁-214₃. Thus, the cell 204₁ may be porated by the TIJ resistor 202₁ and be fed to the transfection chamber 610₁. The reagent 214₁ may be inserted into the cell 204₁. Similarly, the cell 204₂ may be porated by the TIJ resistor 202₂ and be fed to the transfection chamber 610₂. The reagent 214₂ may be inserted into the cell 204₂. The cell 204₃ may be porated by the TIJ resistor 202₃ and be fed to the transfection chamber 610₃. The reagent 214₃ may be inserted into the cell 204₃.

In one example, the transfection chambers 610₁- 610₃ may also include a series of different reagents 214. For example, as the cell 204₁ moves down the transfection chamber 610₁, a side-channel may introduce a different reagent 214 to be inserted into the cell 204. The cell 204 may move further down the transfection chamber 610₁ and another side-channel may introduce another reagent 214 to be inserted into the cell 204, and so forth. Thus, a plurality of different reagents 214 may be inserted into the cell 204 in series, as well as in parallel, or a combination of both. It should be noted that although three transfection chambers 610₁- 610₃ are illustrated in FIG. 6, any number of transfection chambers 610 may be implemented in parallel.

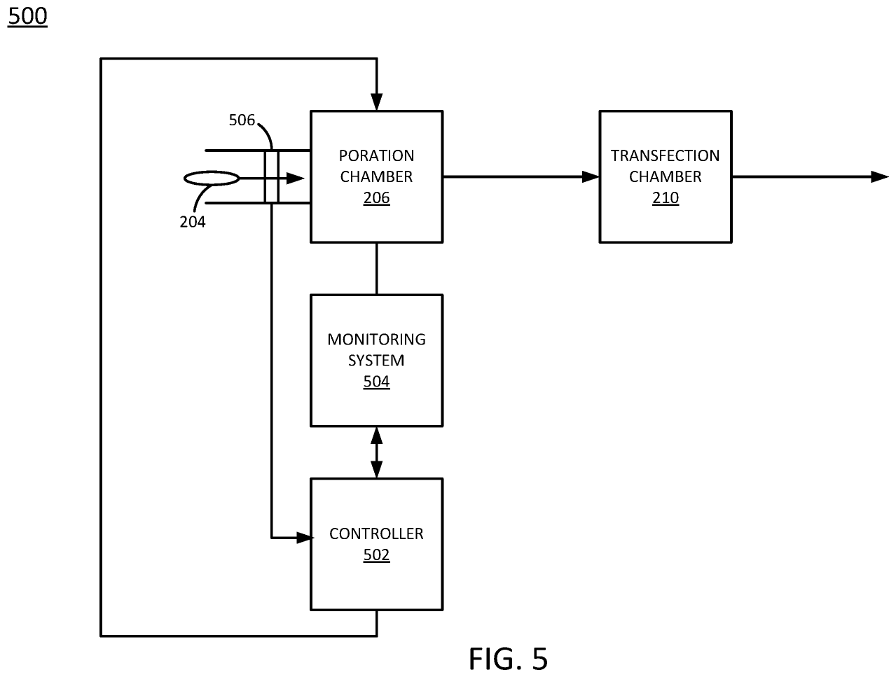

In one example, the poration chamber 206 may include sensors to control activation of the TIJ resistor 202 and provide a feedback loop for controlling the amount of heat applied by the TIJ resistor 202. FIG. 5 illustrates a block diagram of an apparatus 500 to provide thermal cell poration and transfection having a cell monitor and a feedback loop of the present disclosure. In one example, the apparatus 500 may include a poration chamber 206 and a transfection chamber 210 similar to the apparatus 200 illustrated in FIG. 2, and described above. For example, the poration chamber 206 may include a channel 208 having a TIJ resistor 202 to apply heat to a cell 204 and porate the cell 204. The transfection chamber 210 may store a reagent 214 that is inserted into the cell 204 after the cell 204 has been porated.

In one example, the apparatus 500 may also include a controller 502, a sensor 506, and a monitoring system 504. In one example, the controller 502 may be communicatively coupled to the monitoring system 504, the sensor 506, and the TIJ resistors 202 in the poration chamber 206. The controller 502 may be a processor or an external computing device that can control the TIJ resistors 202 in response to signals from the sensor 506 or information collected by the monitoring system 504. It should be noted that the controller 502 may be communicatively coupled to each TIJ resistor 202 when a plurality of TIJ resistors 202 is deployed as illustrated in the arrangements 302 and 304 illustrated in FIG. 3.

In one example, the sensor 506 may transmit a signal to the controller 502 when the cell 204 travels across the sensor 506. The sensor 506 may be an impedance sensor or a capacitive sensor. The sensor 506 may generate and transmit a signal to the controller 502 when an impedance value or a capacitance value changes due to the presence of the cell 204.

The controller 502 may detect the presence of the cell 204 when the signal is received from the sensor 506. In response, the controller 502 may activate the TIJ resistor 202 to heat the cell 204. When no cell 204 is detected, the controller 502 may deactivate the TIJ resistor 202.

Thus, the TIJ resistor 202 may be activated when a cell 204 is detected in the channel 208 rather than continuously activating the TIJ resistor 202. As a result, the sensor 506 may allow the TIJ resistor 202 to be used more efficiently to porate the cell 204 and provide reduced energy usage and lower operating costs.

In one example, the monitoring system 504 may collect information from the cell 204 as the heat is applied to the cell 204. In one example, the type of information that is collected may depend on the type of monitoring system 504 that is implemented. Based on the information that is collected, the controller 502 may adjust the amount of heat that is applied to the cell 204. In one example, the controller 502 may increase or reduce the amount of heat in predetermined increments based on the information collected by the monitoring system 504.

In one example, the monitoring system 504 may include a pair of impedance electrodes. The two impedance electrodes may be located on opposite sides of the TIJ resistor 202 on the top wall 230. The pair of impedance electrodes may have a rectangular shape and may be arranged in parallel, or may have a semi-circular shape and may be arranged to form a circle around the TIJ resistor 202.

While the cell 204 is being heated by the TIJ resistor 202, the impedance electrodes may measure an amount of impedance in the channel 208. As the pores 212 are formed in the cell 204, the amount of impedance may change. The impedance value in the channel 208 when the pores 212 are properly formed in the cell 204 may be pre-defined. The controller 502 may compare the measured amount of impedance to the known impedance value and adjust the amount of heat generated by the TIJ resistor 202 (e.g., increase or lower the amount of heat by adjusting an amount of current that flows through the TIJ resistor 202) until the measured amount of impedance in the channel is within a difference threshold to the known impedance value.

In one example, the monitoring system 504 may include a solution of nanoparticles, an illumination source, and a collection optic. The solution of nanoparticles may be mixed into the fluid containing the cell 204. The nanoparticles may include different sizes and different fluorescence.

The illumination source may provide light on the cell and the collection optic may capture a plurality of images of the cell 204 as the heat is applied to porate the cell. The controller 502 may analyze the plurality of images to measure a rate of migration of particles into the cell 204. The information that is collected may include a pore size and a pore area that is calculated based on the rate of migration of particles into the cell 204. The controller 502 may adjust the amount of heat generated by the TIJ resistor 202 based on the calculated pore size and pore area.

In one example, the monitoring system 504 may include a light source and a photosensor. The light source may apply light to the cell 204 as the cell 204 is being heated. The photosensor may detect the light scattering. The controller 502 may determine whether the pores are being properly formed (e.g., the pore size and pore area) based on a measurement of the light scattering that is measured by the photosensor. For example, data may be collected to know the amount of light scattering that is associated with the desired pore size and pore area. The controller 502 may compare the measured light scattering to the known amount of light scattering to determine if the pores are properly formed. The controller 502 may adjust the amount of heat generated by the TIJ resistor 202 based on the calculated pore size and pore area.

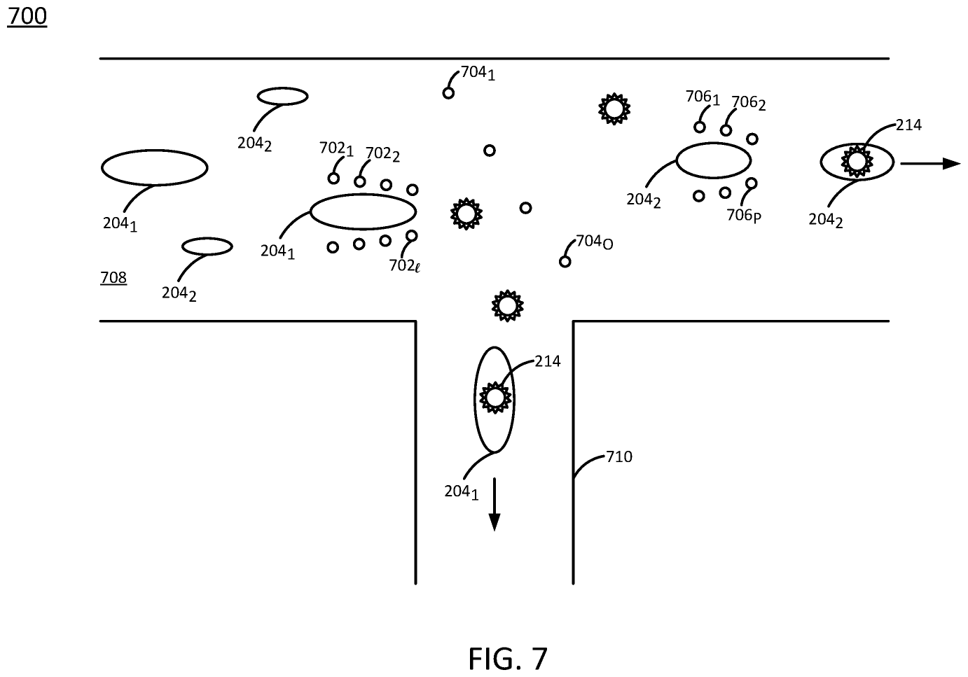

FIG. 7 illustrates a block diagram of a top view of an apparatus 700 to provide thermal cell poration and transfection with a separator. In one example, the apparatus 700 may porate and transfect different sized cells mixed within the same fluid. For example, the fluid may contain a concentration of cells 204₁ and 204₂ that is fed into a channel 708. The cells 204₁ may be larger than the cells 204₂.

In one example, a first portion of the apparatus 700 may include conductive bars 702₁-702_{I} (hereinafter also referred to collectively as conductive bars 702). The conductive bars 702 may be fabricated from a conductive material or metal and may be arranged in a “V” shape. The conductive bars 702 may be coupled to the TIJ resistor 202 (not shown). Thus, as the TIJ resistor 202 generates heat, the conductive bars 702 may conduct the heat through the conductive bars 702 and also apply heat.

In one example, the conductive bars 702 may be arranged in “V” shape to apply heat to the cells 204₁. Notably, the cells 204₂ may be smaller and pass through the conductive bars 702 without being heated.

In one example, separator bars 704₁ to 704₀ (hereinafter also referred to collectively as separator bars 704) may be formed in the channel 708. The separator bars 704 may be fabricated from a non-conductive material and may be used to redirect the larger cells 204₁, while allowing the smaller cells 204₂ to continue downstream to a second set of conductive bars 706₁-706ₚ (hereinafter also referred to collectively as conductive bars 706).

In one example, a reagent 214 may be inserted into the cells 204₁ after being porated by the conductive bars 702. The separator bars 704 may be arranged at an angle to deflect the cells 204₁ containing the reagent 214 into a side-channel 710 to be separated from the cells 204₂.

In one example, the separator bars 704 may be located downstream from the conductive bars 702 at a distance that allows enough time for the cells 204₁ to be transfected with the reagent 214 and to be healed to close the pores formed from the heat applied by the conductive bars 702. The separator bars 704 may also be spaced apart such that the smaller cells 204₂ may flow between the separator bars 704, but such that the larger cells 204₁ are deflected into the side-channel 710.

In one example, the conductive bars 706 may be located downstream from the conductive bars 702 and the separator bars 704. The conductive bars 706 may also be fabricated from a conductive material or metal and may be arranged in a “V” shape. The conductive bars 706 may be coupled to a TIJ resistor 202 that is not shown, similar to the conductive bars 702.

The conductive bars 706 may be arranged such that the cell 204₂ flows through the conductive bars 706 and is heated by contact with the conductive bars 706. The cell 204₂ may be porated and the reagent 214 may be inserted into the cell 204₂.

Although two different sized cells 204₁ and 204₂ are illustrated in FIG. 7, it should be noted that the apparatus 700 may be deployed for any number of different sized cells. For example, additional conductive bars arranged in accordance with the size of the cells may be deployed and additional separator bars may be deployed between each set of conductive bars.

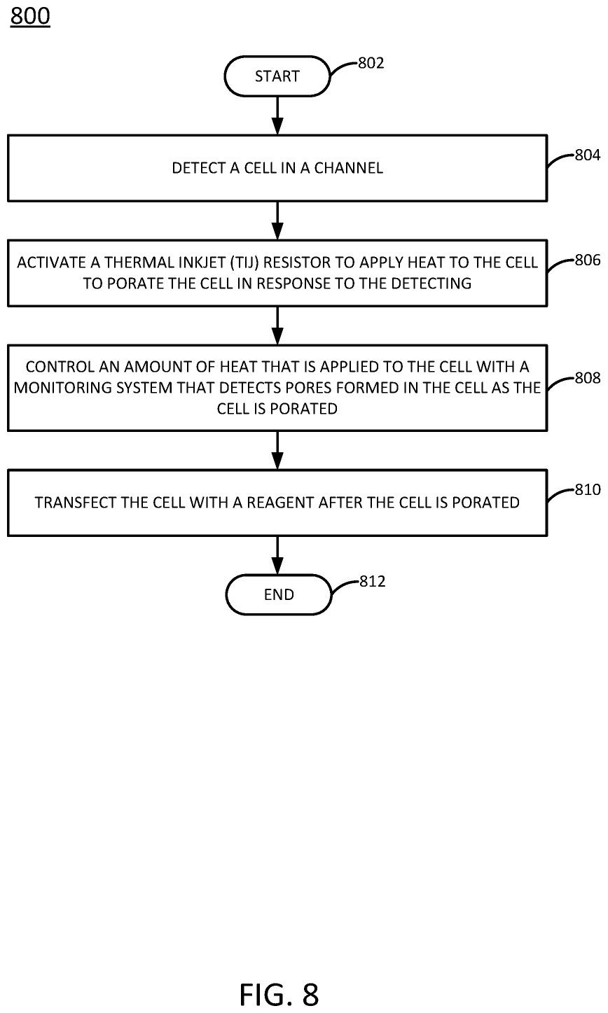

FIG. 8 illustrates a flow diagram of an example method 800 for poration of cells for transfection of the present disclosure. In an example, the method 800 may be performed by the apparatus 500.

At block 802, the method 800 begins. At block 804, the method 800 detects a cell in a channel. For example, when the cell moves past a sensor, a signal may be transmitted to a controller. The controller may determine that a cell is detected in the channel based on the signal from the sensor. The sensor may be an impedance sensor or a capacitive sensor.

At block 806, the method 800 activates a thermal inkjet (TIJ) resistor to apply heat to the cell to porate the cell in response to the detecting. For example, in response to detecting the cell in the channel, the controller may activate the TIJ resistor. When the TIJ resistor is activated, the TIJ resistor may generate heat that is applied to the cell.

In one example, the movement of the cell may be inhibited using various raised surfaces or multiple TIJ resistors as illustrated in FIG. 4 and discussed above. Slowing down the movement of the cell may allow the TIJ resistor to efficiently heat the cell to porate the cell and create pores in the membrane of the cell.

At block 808, the method 800 controls an amount of heat that is applied to the cell with a monitoring system that detects pores formed in the cell as the cell is porated. In one example, the monitoring system may detect whether pores are formed, or may detect more detailed information such as the size of the pores or the pore area. As discussed above, the monitoring system may be a pair of impedance electrodes, a combination of nanoparticles, an illumination source, and a collection optic, or a combination of a light source and a photosensor.

Based on whether pores are formed in the cell or pores are formed with the proper size or area, the controller may adjust the amount of heat that is applied. For example, to generate more heat, the controller may allow more current to pass through the TIJ resistor. To generate less heat, the controller may reduce the amount of current that passes through the TIJ resistor. The amount of heat may be adjusted in predetermined intervals (e.g., in increments of 1 degree, in increments of 5 degrees, in increments of 10 degrees, and the like).

At block 810, the method 800 transfects the cell with a reagent after the cell is porated. After the cell is properly porated, the cell may continue to a transfection chamber. The transfection chamber may store a reagent that is inserted into the cell. At block 812, the method 800 ends.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. An apparatus, comprising:
a channel;
a thermal inkjet (TIJ) resistor to apply heat to a cell in the channel to porate the cell; and
a transfection chamber to store a reagent to be inserted into the cell after the cell is porated.

2. The apparatus of claim 1, wherein the channel comprises a top wall and a bottom wall, and a plurality of TIJ resistors, wherein the plurality of TIJ resistors are arranged on the top wall and the bottom wall to allow the cell to move between the plurality of TIJ resistors.

3. The apparatus of claim 1, wherein the channel comprises a raised surface below the TIJ resistor to constrict the cell.

4. The apparatus of claim 1, wherein the channel comprises a raised surface downstream from the TIJ resistor.

5. The apparatus of claim 1, wherein the TIJ resistor is coupled to a plurality of conductive bars to conduct the heat generated by the TIJ resistor through the plurality of conductive bars, wherein the cell travels through the conductive bars.

6. The apparatus of claim 5, wherein a first set of the plurality of conductive bars arranged to porate a first cell having a first size and a second set of the plurality of conductive bars to porate a second cell having a second size, wherein the second set of the plurality of conductive bars are located downstream form the first set of the plurality of conductive bars.

7. An apparatus, comprising:
a channel;
a thermal inkjet (TIJ) resistor to apply heat to a cell to porate the cell;
a sensor located upstream from the TIJ resistor;
a controller communicatively coupled to the sensor, wherein the controller is to:
detect the presence of the cell in the channel based on a signal received from the sensor; and
activate the TIJ resistor to apply heat in response to detection of the cell in the channel.

8. The apparatus of claim 7, wherein the sensor comprises an impedance sensor or a capacitive sensor.

9. The apparatus of claim 7, further comprising:
a monitoring system to collect information from the cell as the heat is applied to the cell, wherein the controller is communicatively coupled to the monitoring system and the controller is further to:
detect pores formed in the cell based on the information that is collected by the monitoring system; and
control the TIJ resistor to adjust an amount of the heat that is applied to the cell based on detection of the pores formed in the cell.

10. The apparatus of claim 9, wherein the monitoring system comprises an impedance electrode.

11. The apparatus of claim 9, wherein the monitoring system comprises:
a solution of nanoparticles;
an illumination source; and
a collection optic to capture a plurality of images of the cell as the heat is applied to porate the cell, wherein the controller is to analyze the plurality of images to measure a rate of migration of particles into the cell to determine a pore size and a pore area.

12. The apparatus of claim 9, wherein the monitoring system comprises:
a light source to apply light to the cell; and
a photosensor to detect light scattering, wherein the controller is to determine that the pores are formed based on a measurement of the light scattering.

13. The apparatus of claim 7, further comprising:
a plurality of different transfection chamber to store different reagents to be inserted into different cells.

14. A method, comprising:
detecting a cell in a channel;
activating a thermal inkjet (TIJ) resistor to apply heat to the cell to porate the cell in response to the detecting;
controlling an amount of heat that is applied to the cell with a monitoring system that detects pores formed in the cell as the cell is porated; and
transfecting the cell with a reagent after the cell is porated.

15. The method of claim 14, wherein the controlling comprises adjusting the amount of heat based on a pore size and pore area formed in the cell as the cell is porated.
